(19) United States
(12) Patent Application Publication
(10) Pub. No.: US 2013/0245717 A1
(43) Pub. Date: Sep. 19, 2013

(54) Using Alternative Stimulus Waveforms To Improve Pitch Percepts Elicited With Cochlear Implant Systems

(71) Applicant: MED-EL ELEKTROMEDIZINISCHE GERAETE GMBH, Innsbruck (AT)

(72) Inventors: Joshua Stohl, Durham, NC (US); Blake S. Wilson, Durham, NC (US)

(73) Assignee: MED-EL Elektromedizinische Geraete GmbH, Innsbruck (AT)

(21) Appl. No.: 13/786,764

(22) Filed: Mar. 6, 2013

Related U.S. Application Data
(60) Provisional application No. 61/611,122, filed on Mar. 15, 2012.

(51) Int. Cl. A61N 1/36

(52) U.S. Cl. CPC A61N 1/36032
USPC 607/57

(57) ABSTRACT

A cochlear implant system is described which includes an electrode array for implantation in the scala tympani of a cochlea. Electrodes on the outer surface of the electrode array apply electrode stimulation signals to nearby neural tissue. An implantable stimulator module develops the electrode stimulation signals. The electrode stimulation signals have different waveforms. A basal waveform for one or more electrodes at the basal end of the electrode array has the form of a sequence of conventional high-amplitude short-duration electrode stimulation signals. An apical waveform for one or more electrodes at the apical end of the electrode array has the form of a sequence of lower-amplitude longer-duration electrode stimulation signals. The apical waveform is adapted to selectively stimulate peripheral neural processes towards the apical end of the electrode array so as to provide a tonotopic place-pitch response to the electrode stimulation signals.

This application claims priority from U.S. Provisional Patent Application 61/611,122, filed Mar. 15, 2012, which is incorporated herein by reference.

### TECHNICAL FIELD

The present invention relates to cochlear implant (CI) systems that use electrodes placed in the scala tympani (ST) of the cochlea.

### BACKGROUND ART

A human ear normally transmits sounds such as speech sounds as shown in FIG. 1 through the outer ear 101 to the tympanic membrane (eardrum) 102, which moves the bones of the middle ear 103 (malleus, incus, and stapes) that vibrate the oval window membrane of the cochlea 104. The cochlea 104 is a long narrow duct wound spirally about its axis for approximately two and three quarters turns. It includes three chambers along its length: an upper chamber known as the scala vestibuli, a middle chamber known as the scala media, and a lower chamber known as the scala tympani (ST). The cochlea 104 forms an upright spiraling cone with a center called the modiolus where the axons of the auditory nerve 113 reside. These axons project in one direction to the cochlear nucleus in the brainstem and they project in the other direction to the spiral ganglion cells (SGCs) and neural processes peripheral to the cells (hereinafter called peripheral processes) in the cochlea 104. In response to received sounds transmitted by the middle ear 103, sensory hair cells in the cochlea 104 function as transducers to convert mechanical motion and energy into electrical discharges in the auditory nerve 113. These discharges are conveyed to the cochlear nucleus and patterns of induced neural activity in the nucleus are then conveyed to other structures in the brain for further auditory processing and perception.

Hearing is impaired when there are problems in the ability to transmit sound from the external to the inner ears or problems in the transducer function within the inner ear. To improve impaired hearing, auditory prostheses have been developed. For example, when the impairment is related to the operation of the middle ear 103, a conventional hearing aid may be used to provide acoustic stimulation to the auditory system in the form of amplified sound. Or when the impairment is associated with the transducer function in the cochlea 104, a cochlear implant (CI) system can electrically stimulate auditory neural tissue with small currents delivered by multiple electrode contacts (electrodes) distributed along at least a part of the cochlear length (spiral). Arrays of such electrodes normally are inserted into the ST. Alternatively, groups of auditory nerve axons can be stimulated with electrodes placed within the modiolus, or auditory structures in the brain can be stimulated with electrodes placed on or within the structures, for example, on or within the cochlear nucleus. However, these latter placements are together far less than one percent of the ST placements, for the more than 300,000 persons who have received implantable auditory prostheses as of January 2013.

FIG. 1 also shows components of a typical CI system. The system includes an external microphone that provides an audio signal input to an external signal processor 111 which implements a specific signal processing strategy to derive patterns of electrical stimuli from the audio signal input and converts these patterns into a digital data format, such as a sequence of data frames, for transmission from an external transmitter coil 107 to a receiver coil of an implanted stimulator module 108. Besides receiving the processed audio information, the stimulator module 108 also performs additional signal processing such as error correction, pulse formation, etc., and produces electrical stimuli (based on the received data signals) that are sent through an electrode lead 109 to electrodes 110 in an implanted intracochlear electrode array 112 to provide electrical stimulation of cochlear neural tissue associated with the auditory nerve 113. The individual electrodes 110 in the electrode array 112 may excite more or less discrete subpopulations of neurons in the auditory nerve 113 depending on the exact placement of the electrodes 110, the configuration of the electrodes 110 (e.g., so-called monopolar versus bipolar configurations), the survival of excitable neural structures near each of the electrodes 110, and the position of each electrode 110 along the length of the cochlea 104, from the basal end of the cochlea 104 (near the bones of the middle ear 103) to the apical end of the cochlea 104 at the apex of the cochlear spiral. In addition, the waveforms of the stimuli delivered to the electrodes 110 may affect the locus, spatial extent, and synchronicity of neural excitation.

One common electrical stimulation strategy for implantable auditory prostheses is the so called “continuous interleaved sampling” (CIS) strategy introduced by Wilson B S, Finley C C, Lawson D T, Wolford R D, Eddington D K, Rabinowitz W M, “Better Speech Recognition with Cochlear Implants,” Nature, vol. 352, 236-238, July 1991, which is incorporated herein by reference. Signal processing for CIS typically involves the following steps: (1) splitting up of the audio frequency range into spectral bands by means of a filter bank; (2) envelope detection of each filter output signal; (3) instantaneous nonlinear compression of the envelope signal (map law); and (4) modulation of a pulse train for each electrode with the compressed envelope signal for the corresponding band-pass channel.

FIG. 2 shows various functional blocks in a typical CIS processing system. An audio signal is the input to the system, and that audio signal may be sensed by a microphone or provided from another source. This input from the microphone or other source is filtered with a pre-emphasis filter 201 which attenuates strong frequency components in the signal below about 1.2 kHz. Following the pre-emphasis filter 201 are multiple band-pass filters (BPFs) 202 which decompose the output of the pre-emphasis filter into multiple spectral bands. Envelope detectors 203 extract the slowly-varying envelopes of the spectral band signals, for example, by full-wave rectification and low-pass filtering. Compression of the envelopes is performed with a non-linear (e.g., logarithmic) mapping 204 to fit the patient's perceptual characteristics, and the compressed envelope signals are then multiplied with carrier waveforms by modulators 205 to produce non-overlapping biphasic output pulses for the stimulation electrodes (EL-1 to EL-n) implanted in the cochlea. The blocks preceding each electrode, blocks 202, 203, 204, and 205, are alternatively called a channel, a signal channel, a processing channel, a band-pass channel, or a stimulation channel.

CI users can have some difficulty perceiving the electrode stimulation signals according to their position in the cochlea. Reversals and confusions (typically described as differences in pitch, or lack thereof) may produce decrements in CI outcomes as compared to clear identification of all of the electrodes by the user on the basis of different pitches. Recently it has been shown that pitch confusions are disproportionately present towards the apex when stimulating with relatively long electrode arrays that include electrodes in the apical part of the cochlea. Post-mortem analyses of human cochleas have demonstrated that the SGCs in Rosenthal's canal closely approximate the spiral course of the ST up to approximately the second turn, at which point the cell bodies cluster into a so-called “terminal bulb.” Peripheral processes project from the cells in this cluster to the sensory structures in the apical part of the cochlea beyond the second turn. (The sensory structures are generally absent in a deafened cochlea and may be absent in the apical and/or other parts of the cochlea in cases of partial deafness or severe losses in hearing.) If the cells are stimulated with apical electrodes instead of the distal ends of the peripheral processes (which is likely with the short-duration balanced biphasic pulses used in conventional CI systems), then the elicited pitches may be diffuse and also indistinct or relatively indistinct among those apical electrodes because stimulation of each of the electrodes will excite the same undifferentiated cluster of SGCs at about the level of the second turn. The exact population of SGCs excited with stimulation of one apically positioned electrode would be identical or similar to the population excited with any of the other apically positioned electrodes. In contrast, if the distal ends or even the mid portions of the peripheral processes could somehow be stimulated selectively, then the pitches could be less diffuse and far more distinct for the apical electrodes.

### SUMMARY

Embodiments of the present invention are directed to cochlear implant systems which use electrode stimulus signals that have different waveforms. Some of the waveforms may be more effective than others for exciting the distal ends or mid portions of the peripheral neural processes in the cochlea as opposed to exciting the spiral ganglion cells in the cochlea. Balanced biphasic pulses with relatively high amplitudes and short durations for each of the phases are used in conventional cochlear implant systems and those stimuli primarily if not exclusively excite the spiral ganglion cells. In contrast, any of a variety of other stimulus waveforms may be effective in exciting the peripheral processes instead. Selective excitation of the peripheral processes can confer multiple advantages, including but not limited to a greater spatial specificity of excitation and a more stochastic pattern of neural responses, compared with excitation of the spiral ganglion cells or neural structures central to the cells. In the apical part of the cochlea, selective excitation of the peripheral processes also could eliminate or at least ameliorate confusions and reversals among the pitches elicited by the different electrodes at the apical end of the implanted array. Such confusions and reversals are common with the conventional cochlear implant systems using the conventional stimuli. Elimination or reduction of the confusions and reversals with the use of an alternative stimulus waveform—such as balanced biphasic pulses with relatively low amplitudes and long durations for each of the phases—could produce especially large improvements in the hearing abilities of cochlear implant users, including better perception of speech, music, and environmental sounds.

The cochlear implant system includes an electrode array for implantation in the scala tympani of a cochlea. Electrodes on the outer surface of the electrode array apply electrode stimulation signals to nearby neural tissue. An implantable stimulator module develops the electrode stimulation signals. The electrode stimulation signals have different waveforms. A basal waveform for one or more electrodes at the basal end of the electrode array has the form of a sequence of conventional high-amplitude short-duration electrode stimulation signals. An apical waveform for one or more electrodes at the apical end of the electrode array has the form of a sequence of lower-amplitude longer-duration electrode stimulation signals. The apical waveform is adapted to selectively stimulate peripheral neural processes towards the apical end of the electrode array so as to provide a tonotopic place-pitch response to the electrode stimulation signals.

The electrode stimulation signals may be balanced biphasic rectangular pulses having matching duration and absolute amplitudes for each phase. The apical waveform, or the waveform for targeted electrodes elsewhere in the array, may also be asymmetrical, for example, triphasic, pseudo-monophasic, an exponential ramp shape, or an exponentially decaying shape. One or more of these further alternative waveforms may be even more effective than the lower-amplitude and longer-duration balanced biphasic pulses mentioned previously. For any stimulus waveform, a complete balancing of charge would be maintained to preclude the toxic effects on neural tissue of any prolonged accumulation of electrical charge in either the positive or negative direction.

The potential benefits of selective stimulation at the apex would accrue with any of a variety of processing strategies in addition to CIS, as all strategies in current widespread use aim to excite different and discrete subpopulations of neurons along the length of the cochlea with stimulation of the different intracochlear electrodes. In addition, pitch reversals or confusions between or among any of the electrodes in the array may be eliminated or ameliorated with “targeted” manipulations in phase duration (PD) for one or more selected electrodes in the array. Similarly, manipulations in PD may be needed for only a subset of the apical electrodes, e.g., for cases in which some of the electrodes are easily discriminable using the conventional stimuli. In rare cases, all of the apical electrodes may be easily discriminable and no manipulations in PD would be needed.

### BRIEF DESCRIPTION OF THE DRAWINGS

FIG. 1 shows a section view of a human ear with a typical CI system designed to deliver electric stimuli to the inner ear with electrodes placed in the ST.

FIG. 2 shows various functional blocks in a CIS processing system.

FIG. 3 shows various functional blocks in one generic embodiment of the present invention.

### DETAILED DESCRIPTION

Electrical stimulation at apical locations in the ST can elicit pitch percepts that are not discriminable from one another or that are not ordered from high to low with progressively more apical locations. Such confusions and reversals in pitch can occur at other locations, i.e., basal or middle locations, but the confusions and reversals are far more frequent at the apical locations. A lack of discrimination for all pairings of electrodes, or a reversal or reversals in pitch, may degrade hearing abilities—such as speech or music perception—with CI systems.

The predominance of confusions and reversals for the apical locations in the ST is thought to arise from the anatomy of the cochlea. In particular, the SGCs follow the course of cochlear spiral (and the course of the ST) up to about the second turn of the cochlea but then end in a terminal bulb at that level. Peripheral processes projecting from the SCGs within the terminal bulb innervate the sensory structures in the apical part of the cochlea, which includes the final three quarters of a turn. The SGCs are the putative sites of stimulation for electrodes placed within the ST; indeed, the thin peripheral processes are difficult if not impossible to stimulate electrically unless special measures are taken. For conventional CI systems and for excellent survival of the SGCs and peripheral processes, electrodes at basal and middle locations along the length of the ST (and electrode array) may excite subpopulations of SGCs at corresponding positions in Rosenthal's canal, whereas electrodes at apical locations along the length of the ST may stimulate subpopulations of SGCs in the terminal bulb. The subpopulation stimulated by one apical electrode may be essentially identical or highly similar to the subpopulation stimulated by another apical electrode. If so, the pitches elicited with the two electrodes would be expected to be indiscriminable or highly similar as well. In addition, the terminal bulb is not organized tonotopically (i.e., progressively lower pitches for progressively more apical sites of stimulation along the length of the cochlea) but rather is a mixed cluster of SGCs. Thus, even slightly different patterns of excitation within the cluster—resulting from stimulation of different apical electrodes—could lead to idiosyncratic changes in pitch, e.g., pitch reversals.

The SGCs within the terminal bulb are tightly packed and therefore present a more or less uniform target for excitation by relatively distant electrodes, such as the electrodes in the apical part of the ST. In contrast, the distal ends of the peripheral processes projecting from those SGCs are distributed uniformly and broadly along the length of the apical cochlea and follow a strict tonotopic ordering. Those distal ends also are much closer to the electrodes than the SGCs. If the distal ends, or even the mid portions, of the peripheral processes could be excited instead of the SGCs, then the elicited pitches may become distinct or more distinct with stimulation of the apical electrodes and additionally pitch reversals may be eliminated or at least reduced in their severity.

It is known that balanced biphasic pulses with relatively long PDs may preferentially stimulate nerve fibers with relatively small diameters, as compared to the nerve fibers that are stimulated by the short duration pulses used in conventional CI systems. Thus, embodiments of the present invention are directed towards utilizing longer PDs and/or other alternative waveforms in order to stimulate any surviving peripheral processes (which have much smaller diameters than either the SGC somas or distal hillocks) in the apical part of the cochlea rather than the relatively distant SGCs in the terminal bulb. Such an arrangement seeks to obtain an improved representation of place-pitch in the cochlear apex and corresponding improvements in hearing abilities including better perception of speech, music, and environmental sounds.

The pitch associated with a single electrode may be affected by manipulating one or more parameters of alternative waveforms such as the PD of balanced biphasic pulses, presumably due to changes in the site(s) of neuronal excitation. Embodiments of the present invention exploit this effect to help ensure that each electrode elicits a distinct pitch and that the pitches elicited with different electrodes follow the tonotopic mapping of the cochlea. In addition, manipulations in PD or other parameters of alternative waveforms may produce repeatable shifts in pitch, which could be exploited for representing dynamic changes in frequency within a single band-pass channel, for example.

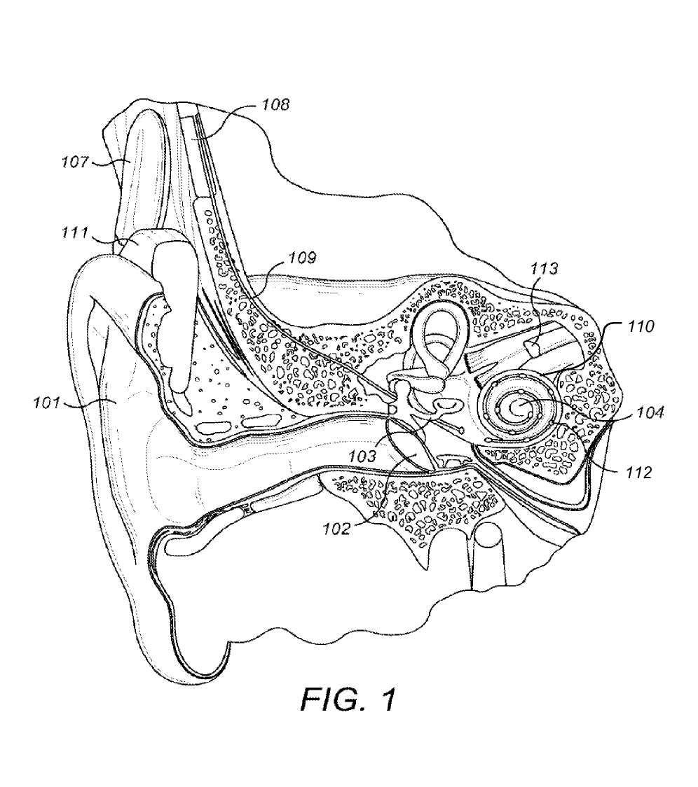

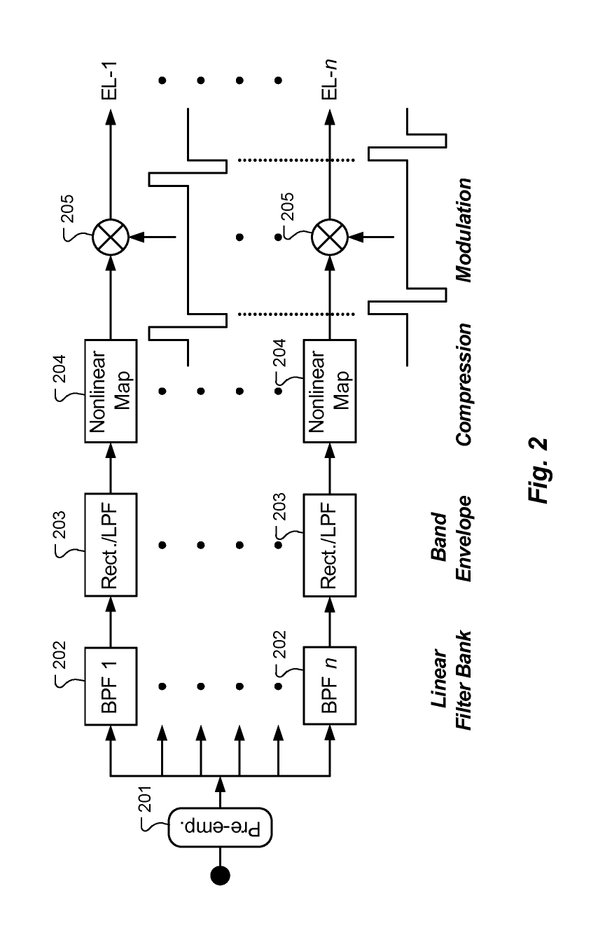

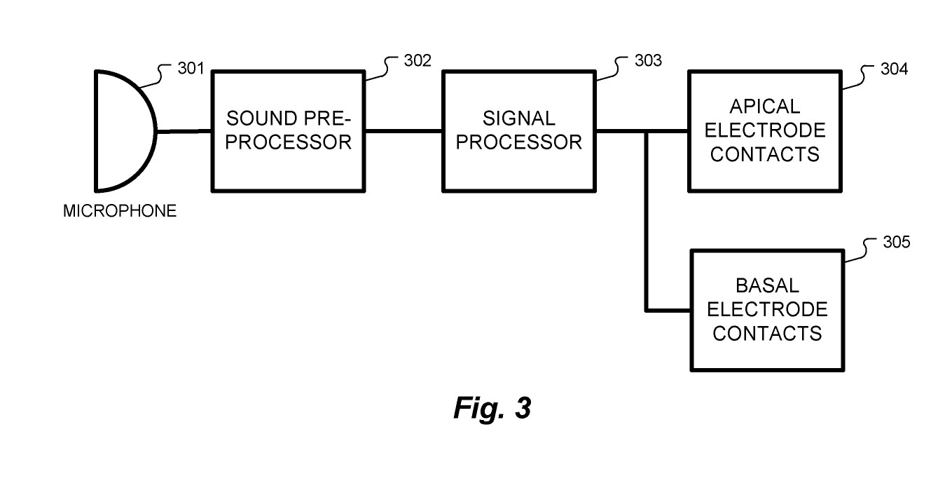

FIG. 3 shows various functional blocks in one generic embodiment of the present invention, which may include components such as the ones shown in FIG. 1 for a CI system. A microphone 301, which is part of the external signal processor 111, senses sound in the environment to generate a representative audio input for subsequent processing. The external signal processor 111 may also contain a sound pre-processor 302 that analyzes the audio input to form a pre-processed audio signal. A signal processor 303 (e.g., in the external signal processor 111 and/or the implanted stimulator module 108) processes the audio signal to produce a representation of the audio frequency information including band-pass envelope characteristics, for example, based on CIS (FIG. 2) or other signal processing strategies for CIs

The signal processor 303 specifies stimuli for all of the utilized electrodes in the implanted array, including the electrodes in the apical and more-basal parts of the array, components 304 and 305, respectively. In this embodiment, the waveform for the apical stimuli is substantially different from the waveform for the stimuli delivered to the other electrodes in the array. For example, low-amplitude and long-duration pulses may be specified for the apical electrodes, whereas the conventional high-amplitude and short-duration pulses may be specified for the remaining electrodes.

Alternative waveforms such as lower amplitude and longer duration pulses may preferentially stimulate thin neural processes as compared to the neural structures stimulated with the conventional high-amplitude and short-duration pulses. Thus, in cases of survival of the peripheral processes, one might therefore expect preferential excitation of fibers at positions closer to apical stimulating electrodes with low-amplitude and long-duration pulses, compared with the non-place-specific stimulation of SGCs in the terminal bulb with high-amplitude and short-duration pulses. In addition, the pitches elicited with preferential stimulation of the peripheral processes might be more distinct and more consistent with the tonotopic positions of the electrodes, as compared with the pitches that would be elicited with stimulation of the SGCs, especially for the apical electrodes.

In some circumstances, the central axons of the auditory nerve may be excited with the conventional stimuli presented at electrodes in the ST. The diameters of the central axons also are much greater than the diameters of the peripheral processes and thus the axons may be stimulated instead of the peripheral processes. Use of an alternative stimulus waveform could shift the site of excitation from the axons to the peripheral processes, which could again improve the tonotopic representation of frequencies, and perceptual distinctions among electrodes, with CI systems.

Waveforms other than rectangular biphasic pulses with long PDs also may be effective in stimulating thin neural processes. Examples of such alternative waveforms include triphasic, pseudo-monophasic, exponentially ramped, or exponentially decaying waveforms. Indeed, stimuli using the pseudo-monophasic and exponentially ramped waveforms have been shown to be especially effective for the selective excitation of small-diameter fibers in motor nerves as described for example in Grill W M and Mortimer J T, “Stimulus Waveforms for Selective Neural Stimulation,” IEEE Engineering in Medicine and Biology Magazine, vol. 14, 375-385, 1995, and in Hennings K L, et al., “Selective Activation of Small-Diameter Motor Fibres Using Exponentially Rising Waveforms: A Theoretical Study,” Medical and Biological Engineering and Computing, vol. 43, 493-500, 2005, which are incorporated herein by reference. These alternative waveforms may be similarly effective for CI systems.

Because even minute direct currents can harm or kill neural tissue, all stimuli delivered through a CI or other neural prosthesis must be balanced for electrical charge over time, e.g., within about 10 milliseconds after the onset of a stimulus waveform. This criterion would need to be met for any of the waveforms proposed in the preceding paragraph. Charge balancing is explicit in charge-balanced biphasic pulses, but is not necessarily explicit for the other waveforms. For those waveforms, a compensating phase or phases may be needed to achieve and assure complete balancing of charge in the specified time frame. In addition, a blocking capacitor between the output of the stimulus source and each intracochlear electrode would guarantee a balancing of charge within the time frame, if an appropriate value in microfarads is selected for the capacitors. The best practice is to assure charge balancing at the output of the stimulus source and before the capacitor, and to include the capacitor to guarantee charge balancing in the unlikely events of a hardware problem in the stimulus source or an error in programming the stimulus source. This approach allows full and predictable control over the stimulus waveform(s) while still providing two levels of protection for the patient.

Selective activation of the peripheral processes also may be helpful in the basal and middle parts of the cochlea. At those locations, one would not necessarily expect consistent shifts in pitch as at the apex, but rather a possibly greater spatial specificity of neural excitation as the target neural structures would be closer to the electrodes. In addition, selective stimulation of the peripheral processes may confer other advantages such as more stochastic patterns of responses within and among auditory neurons compared with stimulation of the SGCs, and those more stochastic patterns may provide closer approximations to the highly stochastic patterns found in normal hearing.

Selective activation in the basal and middle parts of the cochlea may be achieved with the same stimulus waveforms used for selective activation in the apical part of the cochlea, i.e., low-amplitude and long-duration balanced biphasic pulses or any of the other alternative waveforms mentioned previously. The example embodiment of the invention presented in FIG. 3 includes a separation in waveforms for apical versus non-apical electrodes; however, such a separation relates to that embodiment only. Other embodiments include the use of stimulus waveforms that could produce selective activation of the peripheral processes in the basal and middle parts of the cochlea as well. Selective activation of the peripheral processes anywhere in the cochlea may produce more stochastic patterns of responses within and among neurons in the excitation field and also provide a greater spatial specificity of stimulation, compared with stimulation of the SGCs or central axons.

Psychophysical and speech reception studies have been conducted in our laboratory to evaluate in a preliminary way some aspects of the described invention. Balanced biphasic pulses were used exclusively. In broad terms, the results from these studies to date have shown or indicated that: (1) increases in PD beyond the values used in the subjects' conventional CI systems can reliably reduce pitches for apical electrodes; (2) the same or similar increases in PD either do not affect pitch or can produce pitch shifts in either direction for electrodes in the basal or middle portions of the implant array; (3) increases in the PDs for selected electrodes in the implant (usually including the apical electrodes) can increase the number of electrodes for a subject that have significantly different rankings in pitch; and (4) increases in PD for one, some, or all electrodes can produce significant improvements for some subjects in speech reception in noise, particularly for difficult speech items and adverse speech-to-noise ratios, and in the identification of melodic contours. These encouraging (but still preliminary) results are consistent with the ideas that: (1) favorable changes in pitch and distinctions among electrodes can be produced with manipulations in PD especially for the apical electrodes, and (2) those changes can translate to better speech reception and melody identification for at least some users of CI systems. More studies are needed to verify and extend these preliminary results; such studies could include additional subjects, tests, and stimulus waveforms.

Of course, some manipulations in stimulus waveforms may force a reduction in the rate of stimulation across all of the utilized electrodes, e.g., increases in the PDs for balanced biphasic pulses for one or more of the electrodes would produce a reduction in the maximum rate if non-simultaneity of stimulation from one electrode to the next is to be maintained. Some reduction in rate may not produce any deleterious effects. However, a substantial reduction may degrade performance. Substantial reductions can be avoided by increasing PDs for balanced biphasic pulses for only a subset of the electrodes (including only one electrode), and for each of the selected electrodes by increasing the PD just to the point at which the desired change in pitch or other psychophysical attribute is produced. Alternatively, another waveform might be used that would produce the desired change but not require an increase or much of an increase in the overall duration of the stimulus, compared with the short-duration biphasic pulses used in conventional CI systems.

All CI systems in current widespread use support balanced biphasic pulses as stimuli, including balanced biphasic pulses with relatively long PDs. CI systems from MED-EL GmbH are able to produce triphasic pulses as well, whose middle phase is twice the duration of the first and third phase. (The absolute amplitude is the same across the phases.) This waveform is worth considering, in part because it already is available in the MED-EL commercial devices. CI systems from Advanced Bionics AG are capable of generating asymmetric pseudo-monophasic waveforms in which one phase is relatively short and with a high amplitude compared to the opposite (compensating) phase that is long and low in amplitude and has the same charge (the product of amplitude and duration) as the initial phase. And percutaneous implants such as the Ineraid system or the experimental Nucleus Percutaneous devices provide the greatest flexibility with regard to specification and production of stimulus waveforms; those latter systems could be used in studies to evaluate the other alternative waveforms, including variations of the triphasic and pseudo-monophasic waveforms that are not supported by the MED-EL and Advanced Bionics devices, respectively.

Embodiments of the present invention such as those described above offer ease of implementation (e.g., with long-duration biphasic pulses), potential benefits to a large population of CI users, improved salience of place-pitch cues, an additional dimension with which to control the perceived pitch elicited by apical electrode contacts, and power savings through the use of low-amplitude stimuli in some of the embodiments. But longer pulse or other waveform durations can have the effect of reducing the overall stimulation rate of a CI system which may limit the use of certain sound coding strategies. In addition, there also may be increased quantization of amplitude/loudness at low amplitudes that should be addressed. The difference in electrical charge as a result of increasing or decreasing the amplitude of stimulation pulses by a single current step is greater with low amplitude and long duration pulses than with high amplitude and short duration pulses, and this quantization effect at low amplitudes may be a problem with some of the other possible waveforms as well. In addition, the overall approach for increasing the perceptual separations among the apical electrodes is less likely to work well for patients who do not have good enough survival of peripheral processes in that region of the cochlea.

Embodiments of the invention may be implemented in part in any conventional computer programming language such as VHDL, SystemC, Verilog, ASM, etc. Alternative embodiments of the invention may be implemented as pre-programmed hardware elements, other related components, or as a combination of hardware and software components.

Embodiments can be implemented in part as a computer program product for use with a computer system. Such implementation may include a series of computer instructions fixed either on a tangible medium, such as a computer readable medium (e.g., a diskette, CD-ROM, ROM, or fixed disk) or transmittable to a computer system, via a modem or other interface device, such as a communications adapter connected to a network over a medium. The medium may be either a tangible medium (e.g. , optical or analog communications lines) or a medium implemented with wireless techniques (e.g., microwave, infrared or other transmission techniques). The series of computer instructions embodies all or part of the functionality previously described herein with respect to the system. Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies. It is expected that such a computer program product may be distributed as a removable medium with accompanying printed or electronic documentation (e.g. , shrink wrapped software), preloaded with a computer system (e.g., on system ROM or fixed disk), or distributed from a server or electronic bulletin board over the network (e.g. , the Internet or World Wide Web). Of course, some embodiments of the invention may be implemented as a combination of both software (e.g., a computer program product) and hardware. Still other embodiments of the invention are implemented as entirely hardware, or entirely software (e.g., a computer program product).

Although various exemplary embodiments of the invention have been disclosed, it should be apparent to those skilled in the art that various changes and modifications can be made which will achieve some of the advantages of the invention without departing from the true scope of the invention.

What is claimed is:

1. A cochlear implant system comprising:
an electrode array adapted for implantation in the scala tympani of a cochlea, the electrode array having a basal end at an entry into the cochlea and an apical end within the cochlea;
a plurality of electrodes on an outer surface of the electrode array for applying electrode stimulation signals to nearby neural tissue; and
an implantable stimulator module coupled to the electrodes for developing the electrode stimulation signals;
wherein the electrode stimulation signals have a plurality of different waveforms, including:
a. a basal waveform for a plurality of electrodes at the basal end of the electrode array in the form of a sequence of conventional high-amplitude short-duration electrode stimulation signals, and
b. an apical waveform for a plurality of electrodes at the apical end of the electrode array in the form of a sequence of lower-amplitude longer-duration electrode stimulation signals; and
wherein the apical waveform is adapted to selectively stimulate peripheral neural processes towards the apical end of the electrode array so as to provide a tonotopic place-pitch response to the electrode stimulation signals.

2. A system according to claim 1, wherein the apical waveform is asymmetrical.

3. A system according to claim 1, wherein the apical waveform is triphasic.

4. A system according to claim 1, wherein the apical waveform is pseudo-monophasic.

5. A system according to claim 1, wherein the apical waveform is an exponential ramp shape.

6. A system according to claim 1, wherein the apical waveform is an exponentially decaying shape.

7. A system according to claim 1, wherein the electrode stimulation signals include balanced biphasic rectangular pulses having matching duration and absolute amplitudes for each phase.

8. A method of delivering electrode stimulation signals in a cochlear implant system, the method comprising:
applying electrode stimulation signals to nearby neural tissue using an electrode array adapted for implantation in the scala tympani of a cochlea, the electrode array having a basal end at an entry into the cochlea and an apical end within the cochlea;
wherein the electrode stimulation signals have a plurality of different waveforms, including:
a. a basal waveform for a plurality of electrodes at the basal end of the electrode array in the form of a sequence of conventional high-amplitude short-duration electrode stimulation signals, and
b. an apical waveform for a plurality of electrodes at the apical end of the electrode array in the form of a sequence of lower-amplitude longer-duration electrode stimulation signals; and
wherein the apical waveform is adapted to selectively stimulate peripheral neural processes towards the apical end of the electrode array so as to provide a tonotopic place-pitch response to the electrode stimulation signals.

9. A method according to claim 8, wherein the apical waveform is asymmetrical.

10. A method according to claim 8, wherein the apical waveform is triphasic.

11. A method according to claim 8, wherein the apical waveform is pseudo-monophasic.

12. A method according to claim 8, wherein the apical waveform is an exponential ramp shape.

13. A method according to claim 8, wherein the apical waveform is an exponentially decaying shape.

14. A method according to claim 8, wherein the electrode stimulation signals include balanced biphasic rectangular pulses having matching duration and absolute amplitudes for each phase.

15. A cochlear implant system comprising:
an electrode array adapted for implantation in the scala tympani of a cochlea, the electrode array having a basal end at an entry into the cochlea and an apical end within the cochlea;
a plurality of electrodes on an outer surface of the electrode array for applying electrode stimulation signals to nearby neural tissue; and
an implantable stimulator module coupled to the electrodes for developing the electrode stimulation signals;
wherein the electrode stimulation signals for one or more of the electrodes has an alternative waveform differing from conventional high-amplitude short-duration electrode stimulation signals; and
wherein the alternative waveform is adapted to selectively stimulate peripheral neural processes anywhere in the cochlea so as to produce one or more of increased spatial specificity of neural excitation, a shift of elicited pitches, a stochastic pattern of neural responses, and reduced power consumption.

16. A system according to claim 15, wherein the alternative waveform includes balanced biphasic rectangular pulses having matching duration and absolute amplitudes for each phase.

17. A system according to claim 15, wherein the alternative waveform is asymmetrical.

18. A system according to claim 15, wherein the alternative waveform is triphasic.

19. A system according to claim 15, wherein the alternative waveform is pseudo-monophasic.

20. A system according to claim 15, wherein the alternative waveform is an exponential ramp shape.

21. A system according to claim 15, wherein the alternative waveform is an exponentially decaying shape.

22. A method of delivering stimuli to the electrodes in a cochlear implant system, the method comprising:
applying electrode stimulation signals to nearby neural tissue using an electrode array adapted for implantation in the scala tympani of a cochlea, the electrode array having a basal end at an entry into the cochlea and an apical end within the cochlea;
wherein the electrode stimulation signals for one or more of the electrodes has an alternative waveform differing from conventional high-amplitude short-duration electrode stimulation signals; and
wherein the alternative waveform is adapted to selectively stimulate peripheral neural processes anywhere in the cochlea so as to produce one or more of increased spatial specificity of neural excitation, a shift of elicited pitches, a stochastic pattern of neural responses, and reduced power consumption.

23. A method according to claim 22, wherein the alternative waveform includes balanced biphasic rectangular pulses having matching duration and absolute amplitudes for each phase.

24. A method according to claim 22, wherein the alternative waveform is asymmetrical.

25. A method according to claim 22, wherein the alternative waveform is triphasic.

26. A method according to claim 22, wherein the alternative waveform is pseudo-monophasic.

27. A method according to claim 22, wherein the alternative waveform is an exponential ramp shape.

28. A method according to claim 22, wherein the alternative waveform is an exponentially decaying shape.
